(19) United States
(12) Patent Application Publication
(10) Pub. No.: US 2026/0182965 A1
(43) Pub. Date: Jul. 2, 2026

(54) IMAGING A BODY-PART BASED ON SIMULATED BLINKING OF CONTRAST PARTICLES PRESENT THEREIN

(71) Applicant: BRACCO SUISSE SA, Cadempino (CH)

(72) Inventors: Redouane TERNIFI, Plan-les-Ouates (CH); Emmanuel GAUD, Plan-les-Ouates (CH)

(73) Assignee: BRACCO SUISSE SA, Cadempino (CH)

(21) Appl. No.: 19/126,982

(22) PCT Filed: Dec. 21, 2023
(86) PCT No.: PCT/EP2023/087241

§ 371 Date: May 2, 2025

(30) Foreign Application Priority Data
Dec. 22, 2022 (EP) 22215895.8

(51) Int. Cl. A61B 8/00; A61B 8/08; G06T 7/00

(52) U.S. Cl. CPC A61B 8/481; A61B 8/0891; A61B 8/5215; G06T 7/0012; G06T 2207/10132; G06T 2207/20024; G06T 2207/30101

(57) ABSTRACT

A solution for imaging a body-part of a patient is proposed. A corresponding imaging method (700) of ultrasound type comprises generating (742-751) a plurality of masked images from each input image of the body-part by applying corresponding blinking masks (alternating blocking areas and passing areas). Corresponding positions are determined (754-769) of any contrast particles of a contrast agent that are distinguishable (from other contrast particles) in each masked image. Analysis information of the body-part based on the positions of the contrast particles is then output (772-781). A computer program (600) and a corresponding computer program product for implementing the imaging method (700) are also proposed. Moreover, a corresponding computing system (112) and an imaging system (100) comprising it are proposed. A medical method based on the same imaging method (700) is further proposed. WO

### TECHNICAL FIELD

The present disclosure relates to the field of medical imaging. More specifically, this disclosure relates to medical imaging of ultrasound type based on contrast agent.

### BACKGROUND

The background of the present disclosure is introduced hereinafter with the discussion of techniques relating to its context. However, even when this discussion refers to documents, acts, artifacts and the like, it does not suggest or represent that the discussed techniques are part of the prior art or are common general knowledge in the field relevant to the present disclosure.

Medical imaging is well-established technique (in the field of equipment for medical applications) that is used to inspect body-parts of patients by physicians through images providing visual representations thereof (typically, in a substantially non-invasive manner even if the body-parts are not visible directly). Particularly, contrast-enhanced (medical) imaging is based on the administration of a contrast agent to each patient undergoing a (medical) imaging procedure. The contrast agent comprises contrast particles that enhance a contrast of a biological target of interest within the body-part under inspection (for example, a lesion), so as to make it more conspicuous in the images. This facilitates a task of the physicians in several medical applications (for example, in diagnostic applications).

However, medical imaging is diffraction-limited because of an interference of signals used to acquire the images of the body-part. This causes a fundamental limit to a (spatial) resolution of the images, which does not allow distinguishing the representations of points that are closer than a corresponding resolution limit (and particularly the contrast particles appearing as corresponding blurred points). The resolution mainly depends on a wavelength of the signals, with the lower the wavelength the higher the resolution.

Particularly, ultrasound (medical) imaging involves the application of ultrasound waves to the body-part; in this case, an ultrasound contrast agent (USCA), acting as efficient ultrasound reflector, is generally administered to the patient. Corresponding echo signals that are recorded in response to the ultrasound waves are then used to create the images of the body-part. Particularly, the flowing of the contrast particles in blood vessels of the body-part provides a representation of its vascularization.

Ultrasound imaging allows observing the body-part in depth (despite its opacity), with a penetration increasing with the wavelength of the signals. However, the higher the wavelength the lower the resolution. Therefore, when the contrast particles are closer than the resolution limit, they appear overlapped in the images. This does not allow distinguishing blood micro-vessels (wherein the contrast particles flow) that are very close to each other. As a result, the observation of a micro-vasculature of the body-part becomes more and more difficult as its depth within the body-part increases. This hinders the analysis of the micro-vasculature, and particularly of any abnormal alterations thereof that are linked to a broad spectrum of diseases (such as angiogenesis being typical of tumors).

Ultrasound Localization Microscopy (ULM) has been proposed to provide a super-resolution surpassing the resolution limit. Briefly, ULM is based on localization of contrast particles that are isolated. For example, for this purpose the images of the body-part are acquired with a very high (acquisition) rate. The contrast particles being localized are represented with corresponding (smaller) markers at their positions. The positions of the contrast particles are then tracked along the images so as to reconstruct their trajectories within the body-part.

Particularly, different examples of this technique are described in “Short Acquisition time Super-Resolution Ultrasound Microvessel imaging via Microbubble Separation”, Scientific Reports 10:6007 (2020) by C. Huang et al., in “Super-resolution Ultrasound Localization Microscopy through Deep Learning”, IEEE Transactions Medical Imaging, 2021 March, 40 (3):829-839 by R. van Sloun et al., in “Super-resolution Ultrasound Imaging”, Ultrasound in medicine and biology, New York, NY, US, vol. 46, no. 4, 21 Jan. 2020 by Christensen-Jeffries Kirsten et al., in “Deep Learning-Based Super-resolution Ultrasound Speckle Tracking Velocimetry”, Ultrasound in medicine and biology, New York, NY, US, vol. 46, no. 3, 6 Jan. 2020 BY Park Jun Hong et al. and in US-A-2022/240899.

However, ULM requires a relatively low concentration of the contrast agent. In fact, a high concentration of the contrast agent would involve a large amount of overlapped contrast particles in the images that may not be localized individually and then are discarded. As a result, a relatively long acquisition time of the images is required (of the order of hundreds of seconds). Particularly, this is due to the need of detecting several contrast particles to reconstruct the representation of each blood micro-vessel (taking much time to be filled with the contrast agent because of its low flow rate). The administration of the contrast agent may then require non-standard procedures (such as with multiple injections). Moreover, the (long) acquisition time causes artifacts in the images because of an unavoidable motion (of the patient and possibly of a corresponding probe in case of a free-hand scanning), especially due to a breathing of the patient (being the acquisition time higher than a typical breath-holding capability).

In any case, the localization of the contrast particles is discontinuous over time. This reduces an accuracy of the tracking of the contrast particles (for example, adversely affecting the analysis of the micro-vasculature).

All of the above prevents a widespread adoption of ULM in clinical practice.

### SUMMARY

The present invention is set out in the appended claims.

A simplified summary of the present disclosure is herein presented in order to provide a basic understanding thereof; however, the sole purpose of this summary is to introduce some concepts of the disclosure in a simplified form as a prelude to its following more detailed description, and it is not to be interpreted as an identification of its key elements nor as a delineation of its scope.

In general terms, the present disclosure is based on the idea of simulating a blinking of the contrast particles.

Particularly, an aspect provides an imaging method of ultrasound type for imaging a body-part of a patient. The imaging method comprises generating a plurality of masked images from each input image of the body-part by applying corresponding blinking masks (alternating blocking areas and passing areas). Corresponding positions are determined of any contrast particles of a contrast agent that are distinguishable (from other contrast particles) in each masked image. Analysis information of the body-part based on the positions of the contrast particles is then output.

A further aspect provides a computer program for implementing the imaging method.

A further aspect provides a corresponding computer program product.

A further aspect provides a computing system for implementing the imaging method.

A further aspect provides an imaging system comprising the computing system. A further aspect provides a corresponding medical method.

More specifically, one or more aspects of the present disclosure are set out in the independent claims and advantageous features thereof are set forth in the dependent claims, with the wording of all the claims that is incorporated herein verbatim by reference (with any advantageous feature provided with reference to each specific aspect that applies mutatis mutandis to every other aspect).

### BRIEF DESCRIPTION OF THE DRAWINGS

The solution of the present disclosure, as well as further features and the advantages thereof, will be better understood with reference to the following detailed description thereof, provided purely by way of a non-restrictive indication, to be read in conjunction with the accompanying drawings (wherein, for the sake of simplicity, corresponding elements are denoted with equal or similar references and their explanation is not repeated, and the name of each entity is generally used to denote both its type and its attributes, like value, content and representation). Particularly:

FIG. 1 shows a pictorial representation of an imaging system that may be used to practice the solution according to an embodiment of the present disclosure,

FIG. 2 shows an exemplary image that may be obtained with this imaging system,

FIG. 3 shows an exemplary imaging procedure based on a ULM technique known in the art,

FIG. 4A-FIG. 4D show the general principles of the solution according to an embodiment of the present disclosure,

FIG. 5A-FIG. 5D show different examples of blinking masks that may be used to implement the solution according to an embodiment of the present disclosure,

FIG. 6 shows the main software components that may be used to implement the solution according to an embodiment of the present disclosure,

FIG. 7A-FIG. 7B show an activity diagram describing the flow of activities relating to an implementation of the solution according to an embodiment of the present disclosure, and

FIG. 8 shows a comparative example of in-vitro imaging procedures known in the art and according to an embodiment of the present disclosure.

### DETAILED DESCRIPTION

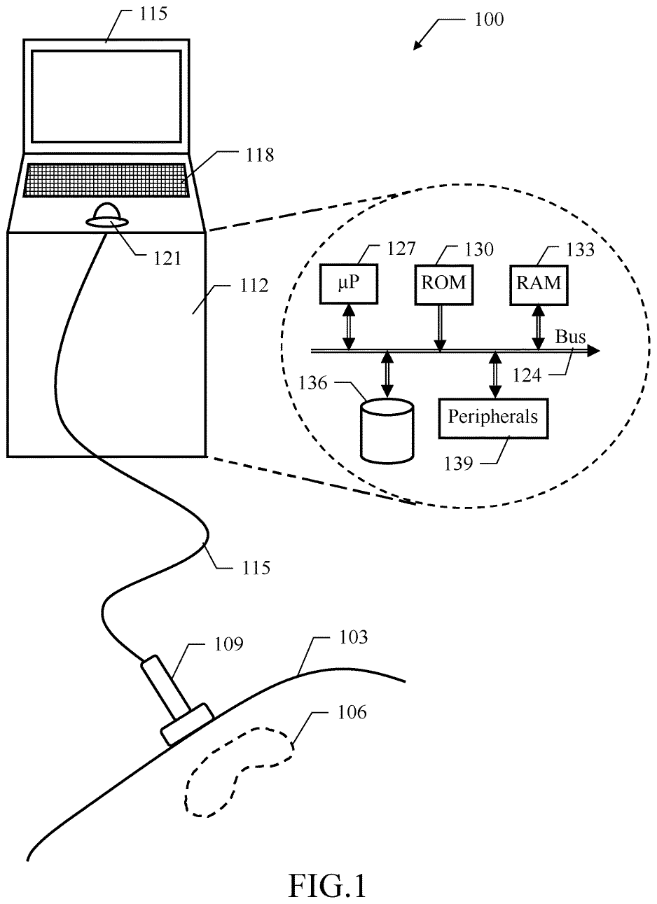

With reference to FIG. 1, a pictorial representation is shown of an imaging system 100 that may be used to practice the solution according to an embodiment of the present disclosure.

The imaging system 100 allows imaging a scene comprised in a field of view thereof (defined by a part of the world within a solid angle to which the imaging system 100 is sensitive). Particularly, the imaging system 100 is an ultrasound scanner that is used in (medical) imaging procedures to assist a physician (such as for diagnostic applications). In this case, the scene relates to a patient 103 to whom an (ultrasound) contrast agent, comprising contrast particles acting as efficient ultrasound reflector, has been administered. Particularly, the scene comprises a body-part 106 of the patient 103 to be inspected (for example, for discovering or monitoring a lesion, such as a tumor).

The ultrasound scanner 100 comprises an imaging probe (or transducer) 109 for acquiring images of its field of view and a central unit 112 for controlling its operation. For this purpose, the imaging probe 109 is coupled with the central unit 112, for example, via a corresponding flexible cable 115.

The imaging probe 109 is of hand-held type, being sized so as to be held by a single hand of the physician. For example, I imaging probe 109 operates in a pulse-echo mode, wherein it alternatively transmits pulses of ultrasound waves and receives echo signals resulting from their reflection. For this purpose, the imaging probe 109 has an array of sensors (not shown in the figure), which serves to generate the ultrasound waves and to receive the corresponding echo signals, for example, with a phased-array arrangement. The imaging probe 109 works at a frequency determining a penetration of the imaging procedure within the patient 103 (with the higher the frequency, and then the lower a corresponding wavelength, the lower the penetration). For example, the ultrasound waves may have a center frequency spanning from 1.5 MHz to 30 MHz, which allows observing the patient up to a depth of about 300 mm and 15 mm, respectively.

The central unit 112 is provided with a monitor 115 (for displaying the images relating to each imaging procedure that is in progress) and one or more input units, such as a keyboard 118 with a trackball 121 (for controlling operation of the ultrasound scanner 100). The central unit 112 comprises several components that are connected among them through a bus structure 124. Particularly, a microprocessor (μP), or more, 127 provides a logic capability of the central unit 112. A non-volatile memory (ROM) 130 stores basic code for a bootstrap of the central unit 112 and a volatile memory (RAM) 133 is used as a working memory by the microprocessor 127. The central unit 112 has a mass-memory 136 for storing programs and data, for example, a Solid-State-Disk (SSD). Moreover, the central unit 112 comprises a number of controllers 139 for peripherals, or Input/Output (I/O) units, comprising the imaging probe 109, the monitor 115, the keyboard 118 and the trackball 121, and (not shown in the figure) a network adapter for connecting the ultrasound scanner 100 to a network, a drive for reading/writing removable storage units (such as USB keys) and so on.

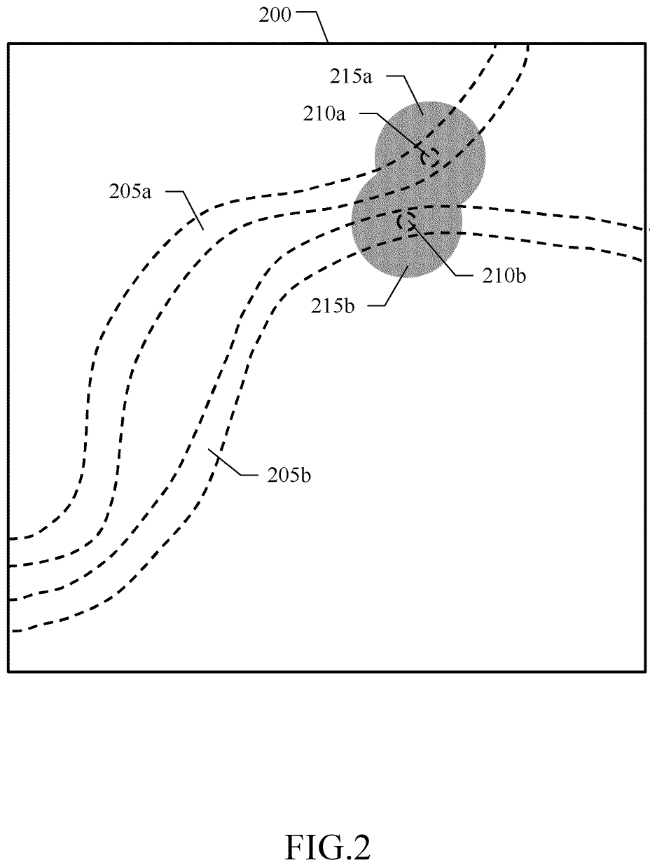

With reference to FIG. 2, an exemplary image 200 is shown that may be obtained with this imaging system.

Particularly, during each imaging procedure a sequence of (input) images are provided; for example, the images are acquired from the body-part of the patient after administering the contrast agent thereto, and then they are filtered to remove a contribution of a tissue thereto. Therefore, the images substantially represent only the contrast particles that are present in the body-part under inspection. Each image is always a blurred representation of the contrast particles due to a resolution limit of the ultrasound scanner (with the higher the frequency, and then the lower the corresponding wavelength, the higher the resolution), for example, spanning from 500 um at 1.5 MHz to 25 um at 30 MHz. More specifically, each contrast particle appears as a blurred point given by its convolution with a Point Spread Function (PSF) of the ultrasound scanner (defining a response thereof to a point source). Therefore, the contrast particles are overlapped when they are closer than the resolution limit.

For example, the image 200 relates to a body-part with two blood micro-vessels 205a and 205b (having a diameter of the order of 5-20 μm). The blood micro-vessels 205a,205b are not visible in the image 200 (only representing the contrast agent); therefore, they have been added in dashed lines to the image 200 to show their position in the body-part, wherein they are closer than the resolution limit. The blood micro-vessels 205a,205b contain contrast particles (with a lower diameter, such as 1-5 μm); particularly, the blood micro-vessel 205a contains a contrast particle 210a and the blood micro-vessel 205b contains a contrast particle 210b, in dashed lines as well since not distinguishable in the image 200. The contrast particles 210a and 210b are instead represented in the image 200 as corresponding blurred points 215a and 215b, respectively. The contrast particles 210a,210b as well are closer than the resolution limit, so that their blurred points 215a,215b are overlapped. As a consequence, it would not be possible to distinguish the blood micro-vessels 205a,205b in the image 200 with conventional ultrasound imaging techniques.

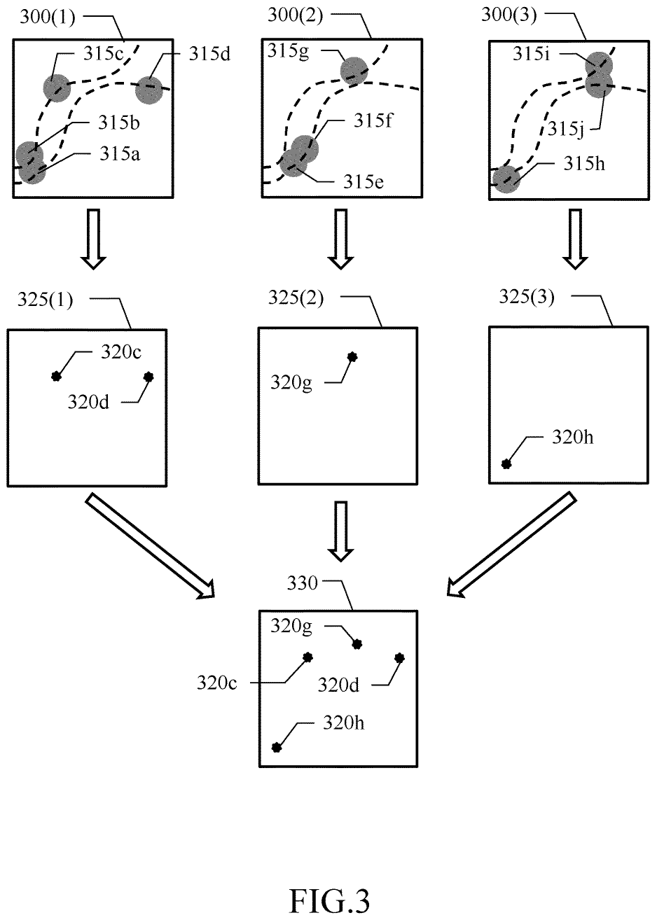

With reference now to FIG. 3, an exemplary imaging procedure is shown based on a ULM technique known in the art.

In this case, any contrast particles (represented by the corresponding blurred points in the images) are localized so as to determine their positions when it is possible, i.e., when the contrast particles are isolated; the (isolated) contrast particles are then represented with markers corresponding to their positions (smaller than their representation in the images, i.e., smaller than the resolution limit).

For example, three (input) images 300(1), 300(2) and 300(3) being obtained over time are shown of the contrast particles present in the same body-part of FIG. 2 above. The image 300(1) contains (the representations of) four contrast particles 315a, 315b, 315c and 315d, the image 300(2) contains (the representations of) three contrast particles 315e, 315f and 315g, and the image 300(3) contains (the representations of) three contrast particles 315h, 315i and 315j. In the image 300(1), the contrast particles 315c and 315d being isolated are localized so as to determine their positions, represented with corresponding markers 320c and 320d, respectively, in an image 325(1); the contrast particles 315a and 315b being overlapped are instead discarded. In the image 300(2), the contrast particle 315g being isolated is localized so as to determine its position, represented with a corresponding marker 320g in an image 325(2); the contrast particles 315e and 315f being overlapped are instead discarded. In the image 300(3), the contrast particle 315h being isolated is localized so as to determine its position, represented with a corresponding marker 320h in an image 325(3); the contrast particles 315i and 315j being overlapped are instead discarded.

The (positions of the) contrast particles 320c, 320d, 320g and 320h being localized over time are cumulated into a map 330 (tracking them for reconstructing the trajectories being followed by the contrast particles within the body-part along its blood micro-vessels). However, in this case many (overlapped) contrast particles are discarded. Therefore, a relatively low concentration of the contrast agent is required to increase a likelihood of having contrast particles that are isolated (for example, of the order of 103-104 contrast particles per mL), with a corresponding increase of an acquisition time of the images.

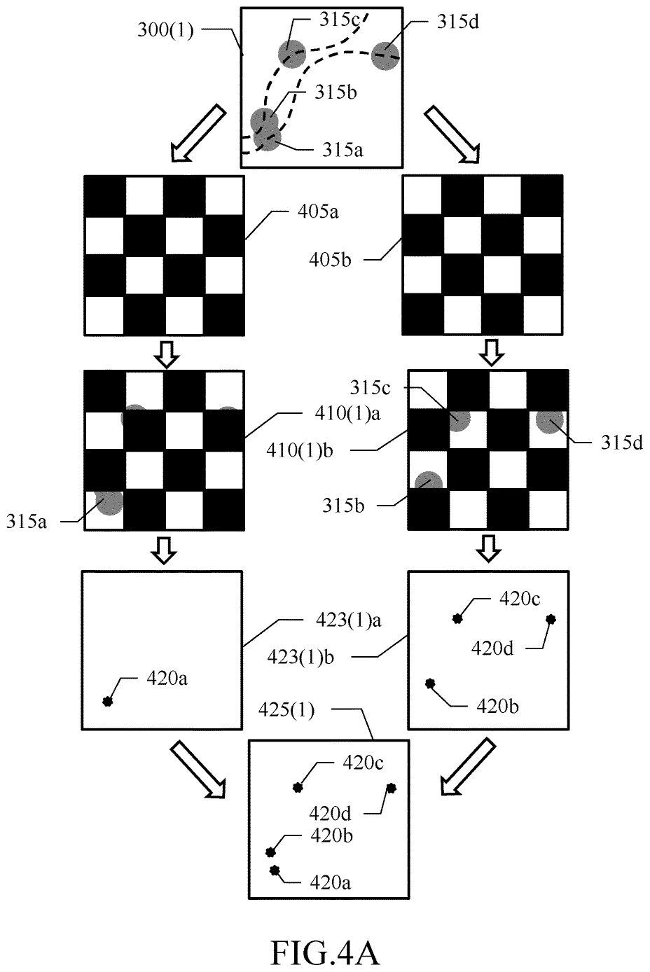

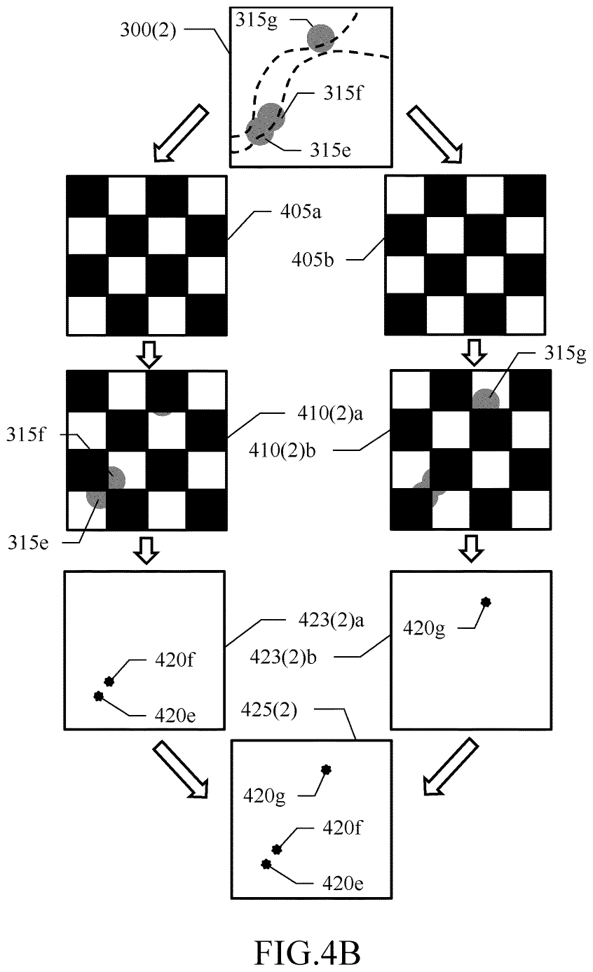

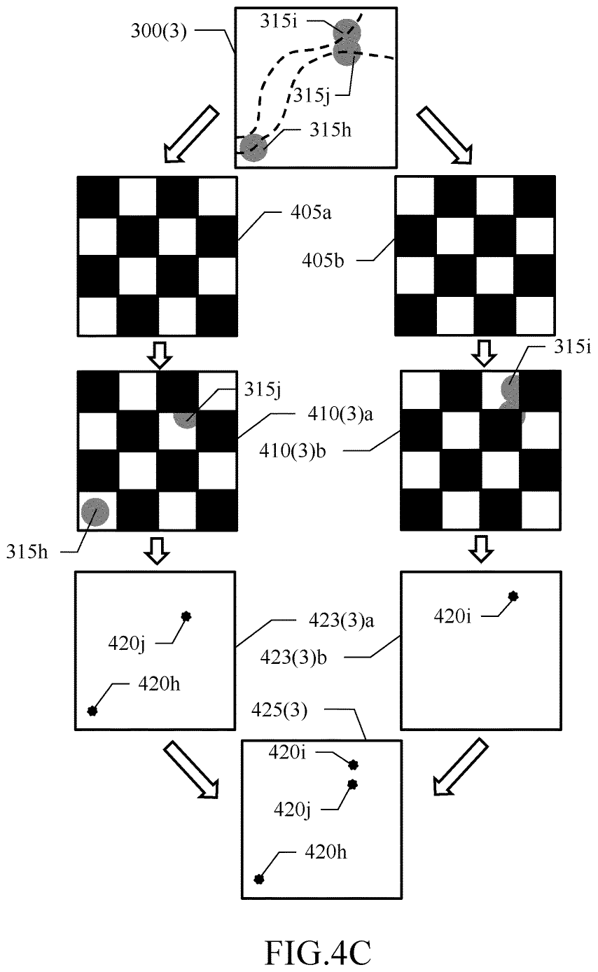

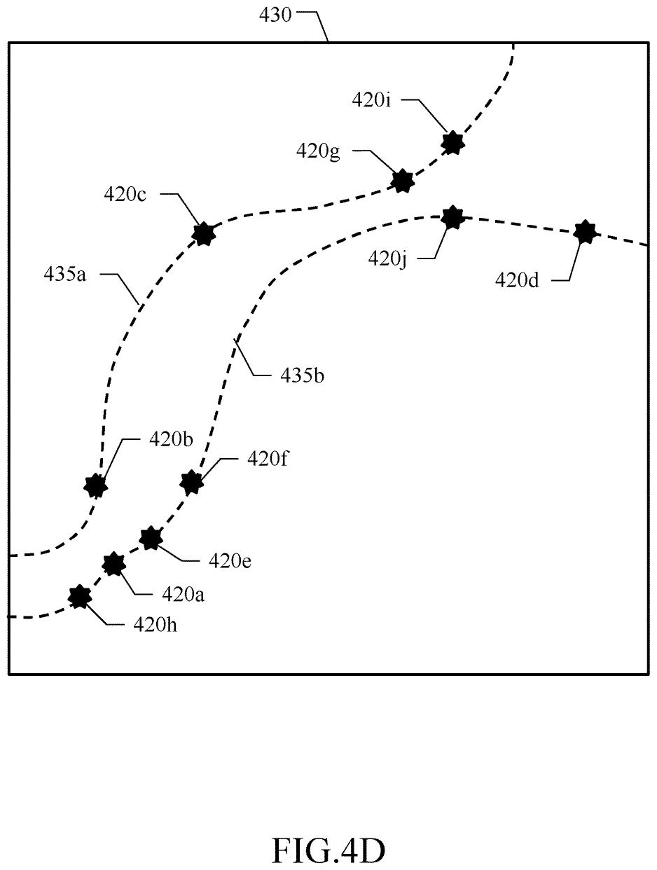

With reference now to FIG. 4A-4D, the general principles are shown of the solution according to an embodiment of the present disclosure.

In this case, a blinking of the contrast particles is simulated. Particularly, a plurality of blinking masks is provided. Each blinking mask alternates a plurality of blocking areas and a plurality of passing areas. When the blinking mask is applied to an analysis region of each image (for example, equal to the whole image), the blocking areas mask a corresponding portion of the image (resetting it to be indicative of no echo signal) whereas the passing areas do not mask a corresponding portion of the image (maintaining it unchanged). In a specific embodiment, the blinking masks are defined so that the passing areas of all the blinking masks do not mask the whole analysis region of the images (thereby ensuring that the application of all the blinking masks to each image maintains its analysis region unchanged overall, with every point of the analysis region that is not masked by at least one blinking mask). Each image is masked by applying the blinking masks thereto, thereby making it sparser (the representation of) the contrast particles being present therein. Any contrast particles being distinguishable in the (masked) images are localized so as to determine their positions. The distinguishable contrast particles comprise the ones that are already isolated from the other contrast particles in the (original) images. However, the distinguishable contrast particles further comprise the ones that, although overlapped with other contrast particles in the (original) images, now have a degree of overlapping that is substantially reduced by the application of the blinking masks; for example, a total extent of each of these contrast particles and a residual part of the other overlapping contrast particles is lower than 1.4-1.6 times (such as 1.5 times) an extent of each contrast particle.

For example, the figures show the application of this technique to the same images of FIG. 3 above.

Starting from FIG. 4A, two blinking masks 405a and 405b are provided (in this case with the same size as the images to which they are to be applied). The blinking masks 405a,405b are divided into alternated blocking areas and passing areas with a square shape, being represented in black and white, respectively, in the figure. The blinking masks 405a, 405b are complementary (with the blocking areas and the passing areas of the blinking mask 405a corresponding to the passing areas and to the blocking areas, respectively, of the blinking mask 405b). The blinking masks 405a and 405b are applied to the image 300(1) of FIG. 3, so as to obtain corresponding (masked) images 410(1)a and 410(1)b, respectively. Therefore, the image 410(1)a maintains the contrast particle 315a and the image 410(1)b maintains the contrast particles 315b, 315 c, 315 d that are now all distinguishable. In the image 410(1)a, the (distinguishable) contrast particle 315a is localized so as to determine its position, represented with a corresponding marker 420a, in a (partial) image 423(1)a. In the image 410(1)b, the (distinguishable) contrast particles 315b, 315c and 315d are localized so as to determine their positions, represented with corresponding markers 420b, 420c and 420d, respectively, in a (partial) image 423(1)b. The images 423(1)a and 423(1)b are then combined into a (localization) image 425(1) wherein (the positions of) all the contrast particles 420a-420d are represented.

Moving to FIG. 4B, the blinking masks 405a and 405b are applied to the image 300(2) of FIG. 3, so as to obtain corresponding (masked) images 410(2)a and 410(2)b, respectively. Therefore, the image 410(2)a maintains the contrast particles 315e, 315f and the image 410(2)b maintains the contrast particle 315g that are now all distinguishable. In the image 410(2)a, the (distinguishable) contrast particles 315e and 315f are localized so as to determine their positions, represented with corresponding markers 420e and 420f, respectively, in a (partial) image 423(2)a. In the image 410(2)b, the (distinguishable) contrast particle 315g is localized so as to determine its position, represented with a corresponding marker 420g in a (partial) image 423(2)b. The images 423(2)a and 423(2)b are then combined into a (localization) image 425(2) wherein (the positions of) all the contrast particles 420e-420g are represented.

Moving to FIG. 4C, the blinking masks 405a and 405b are applied to the image 300(3) of FIG. 3, so as to obtain corresponding (masked) images 410(3)a and 410(3)b, respectively. Therefore, the image 410(3)a maintains the contrast particles 315h, 315j and the image 410(3)b maintains the contrast particle 315i that are now all distinguishable. In the image 410(3)a, the (distinguishable) contrast particles 315h and 315j are localized so as to determine their positions, represented with corresponding markers 420h and 420j, respectively, in a (partial) image 423(3)a. In the image 410(3)b, the (distinguishable) contrast particle 315i is localized so as to determine its position, represented with a corresponding marker 420i in a (partial) image 423(3)b. The images 423(3)a and 423(3)b are then combined into a (localization) image 425(3) wherein (the positions of) all the contrast particles 420h-420i are represented.

Moving to FIG. 4D, (analysis) information of the body-part, based on the (positions of the) contrast particles 420a-420j being localized, is then output. For example, the positions of the contrast particles 420a-420j are tracked for reconstructing corresponding trajectories (followed by them within the body-part along its blood micro-vessels) into a (cumulated) map 430. In this case, a trajectory 435a is reconstructed being followed by a contrast particle represented by the markers 420b, 420c, 420g and 420i, and a trajectory 435b is reconstructed being followed by another contrast particle represented by the markers 420h, 420a, 420e, 420f, 420j and 420d (corresponding to the blood micro-vessels 205a and 205b, respectively, of FIG. 2). A super-resolution image of the body-part (not shown in the figure) may then be generated showing a representation of the trajectories 435a and 435b (and then of a micro-vasculature of the body-part).

The above-mentioned solution significantly increases a number of the contrast particles that are (artificially) isolated in the images, so that the contrast particles may be localized even if they are overlapped. This solution may then be applied with a relatively dense (moderate/high) concentration of the contrast agent (for example, higher than 10⁵-10⁶ contrast particles per mL). As a result, the acquisition time of the images is significantly reduced. This may allow administering the contrast agent with standard procedures (such as with a single bolus injection). Moreover, any motion during the acquisition time may be reduced (for example, by asking the patient to hold breath), thereby limiting corresponding artifacts.

A quality of the obtained results is then significantly improved. Particularly, the localization of the contrast particles is more continuous, thereby facilitating their tracking. This is particularly beneficial for analyzing the micro-vasculature of the body-part, providing valuable information about its morphological and/or hemodynamic characteristics that are indicative of several lesions (such cancer, arteriolosclerosis, diabetes and so on).

The above-mentioned result is achieved in a relatively simple way, with a computational complexity that may be readily adapted according to contingent needs (by changing the blinking masks accordingly).

All of the above fosters a widespread adoption of the proposed technique in clinical practice. Particularly, it is possible to surpass the resolution limit by more than one order of magnitude (for example, allowing distinguishing contrast particles that are spaced apart by about 50 μm at 1.5 MHz down to 2.5 μm at 30 MHz).

The proposed solution facilitates a task of the physician in several medical applications (for example, in diagnostic applications for discovering/monitoring lesions, in therapeutic applications for delineating lesions to be treated and in surgical applications for recognizing margins of lesions to be resected).

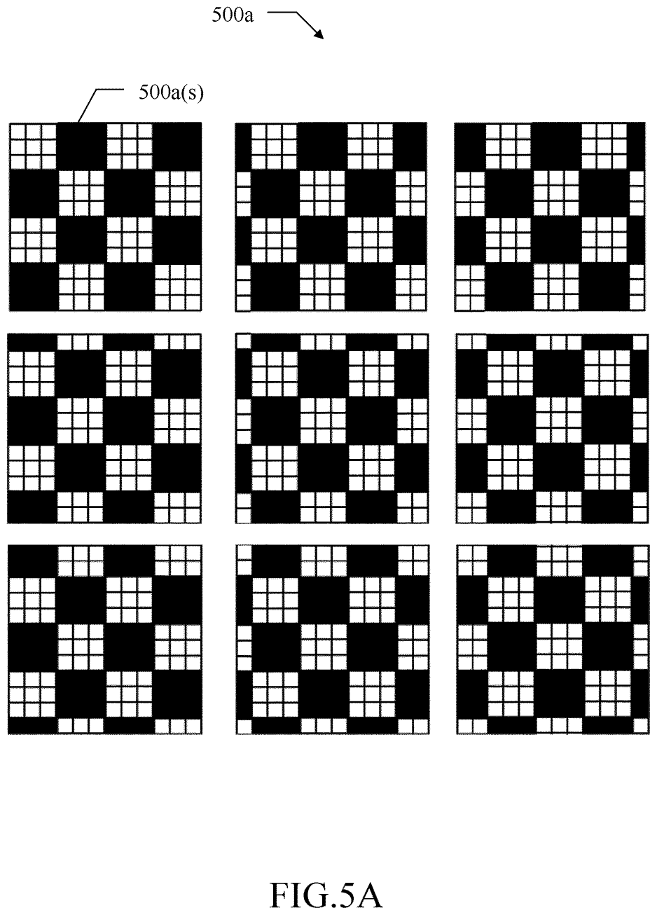

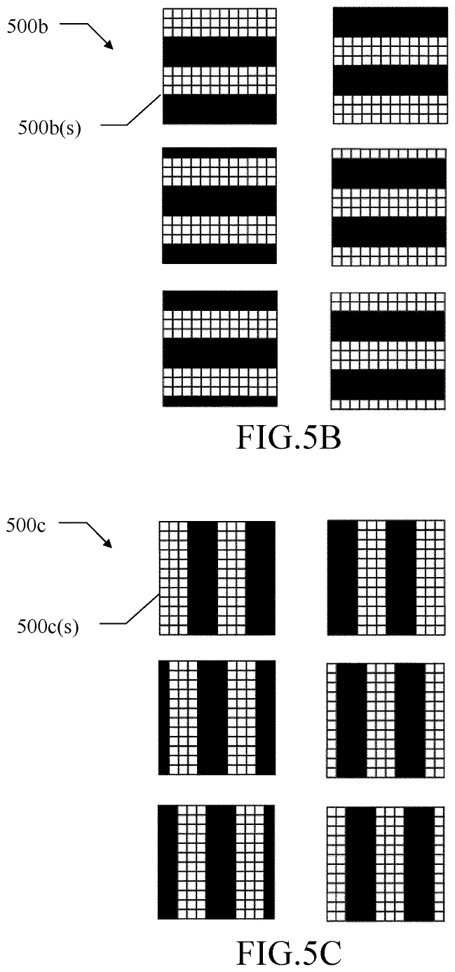

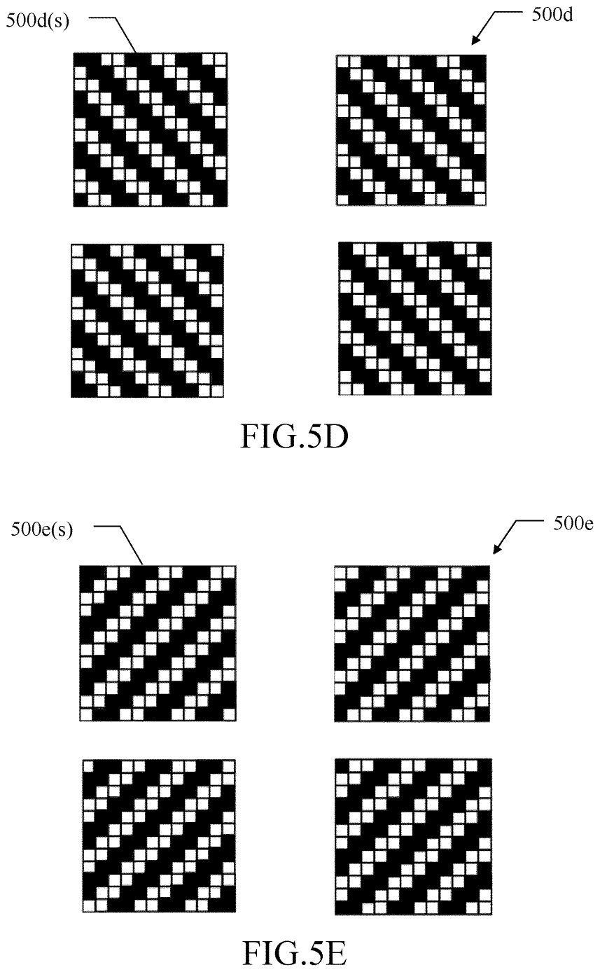

With reference now to FIG. 5A-FIG. 5D, different examples are shown of blinking masks that may be used to implement the solution according to an embodiment of the present disclosure.

Each blinking mask comprises a matrix of cells (arranged in corresponding rows and columns) that are used to mask (black cells) or not to mask (white cells) corresponding basic picture elements (such as pixels) of the images, which represent corresponding locations of the body-part. In general, the passing areas of each blinking mask (and then its blocking areas as well) have an extent, in terms of number of cells, that is defined according to the opposite requirements of high capability of isolating the contrast particles (smaller passing areas that facilitate masking other contrast particles being overlapped) and low computational time (bigger passing areas that reduce the number of blinking masks required to have the whole analysis region of the images not masked overall). For example, the passing areas have an extent with at least one dimension equal to a multiple of an extent of the representation of each contrast particle in the images (as defined by the PSF of the ultrasound scanner) by a factor F, with F=0.5-1.5, preferably F=0.8-1.2 and still more preferably F=0.9-1.1, such as F=1.0. Therefore, when the size of the contrast particles is lower than the resolution limit, the extent of the representation of each contrast particle in the images corresponds to the resolution limit of the ultrasound scanner (about half the wavelength of the signals used to acquire the images, given by a speed of the ultrasound waves in the body-part, such as 1,504 m/s, divided by their frequency); in this case, at least one dimension of the passing areas may be set to a number of cells corresponding to F·λ/2 (i.e., from 0.25·λ to 0.75·λ, preferably λ/2 in the example at issue, wherein λ is the wavelength), such as of the order of 5-15 cells.

The blinking masks may comprise one or more mask sets for corresponding mask patterns. Each mask set comprises a plurality of blinking masks that have the blocking areas and the passing areas featuring the mask pattern of the mask set with corresponding displacements along one or more shifting directions. The blinking masks of the mask set may have been generated from a seed (blinking) mask, which is shifted (in a wrap-around manner) along each shifting direction by a shifting stride repeatedly until returning to the seed mask. In this case as well, the shifting stride is defined according to the opposite requirements of high capability of isolating the contrast particles (lower shifting stride) and low computational time (higher shifting stride); for example, when the blocking/passing areas have a same width along the shifting direction, a shifting stride equal to 1 cell requires a number of shifts equal to the width of the blocking/passing areas (higher isolating capability but higher computational time), whereas a shifting stride equal to the width of the blocking/passing areas requires a single shift (lower computational time but lower isolating capability).

Starting from FIG. 5A, very simple blinking masks 500a are shown with a chessboard pattern. Particularly, a seed mask 500a(s) is defined to comprise (horizontally/vertically) alternated blocking areas and passing areas formed by corresponding squares with a common extent. The other blinking masks 500a are generated from the seed mask 500a(s) by shifting it in a wrap-around manner along one dimension (rows or columns) by a selected shifting stride. If the shifting stride is lower than the extent of the passing areas along this dimension, each (seed/shifted) blinking mask 500a is further shifted along the other dimension (columns or rows) by a selected shifting stride (either the same or different with respect to above). For example, FIG. 5A shows 9 blinking masks 500a each of 12×12 cells. The seed mask 500a(s) has the blocking/passing areas formed by squares of 3×3 cells. The other blinking masks 500a are generated from the seed mask 500a(s) by shifting it in a wrap-around manner along the rows and along the columns by 1 cell (moving rightwards and downwards, respectively).

Moving to FIG. 5B, very simple blinking masks 500b are shown with a horizontal-strips pattern. Particularly, a seed mask 500b(s) is defined to comprise alternated blocking areas and passing areas formed by corresponding horizontal strips with a common width. The other blinking masks 500b are generated from the seed mask 500b(s) by shifting it in a wrap-around manner along the rows by a selected shifting stride. For example, FIG. 5B shows 6 blinking masks 500b each of 12×12 cells. The seed mask 500b(s) has the blocking/passing areas formed by horizontal strips of 3x 12 cells. The other blinking masks 500b are generated from the seed mask 500b(s) by shifting it in a wrap-around manner along the rows by 1 cell (moving downwards).

Moving to FIG. 5C, very simple blinking masks 500c are shown with a vertical-strips pattern. Particularly, a seed mask 500c(s) is defined having alternated blocking areas and passing areas formed by corresponding vertical strips with a common width. The other blinking masks 500c are generated from the seed mask 500c(s) by shifting it in a wrap-around manner along the columns by a selected shifting stride. For example, FIG. 5C shows 6 blinking masks 500c each of 12×12 cells. The seed mask 500c(s) has the blocking/passing areas formed by vertical strips of 12×3 cells. The other blinking masks 500c are generated from the seed mask 500c(s) by shifting it in a wrap-around manner along the columns by 1 cell (moving rightwards).

Moving to FIG. 5D, very simple blinking masks 500d are shown with a downward-diagonal-strips pattern. Particularly, a seed mask 500d(s) is defined having alternated blocking areas and passing areas formed by corresponding downward-diagonal strips (extending downwards moving from left to right) with a common width. The other blinking masks 500d are generated from the seed mask 500d(s) by shifting it in a wrap-around manner (extending or reducing the downward-diagonal strips accordingly) along an upward-diagonal direction (extending upwards moving from left to right) by a selected shifting stride. For example, FIG. 5D shows 4 blinking masks 500d each of 12×12 cells. The seed mask 500d(s) has the blocking/passing areas formed by downward-diagonal strips with a width of 2 cells. The other blinking masks 500d are generated from the seed mask 500d(s) by shifting it in a wrap-around manner along the upward-diagonal direction by 1 cell (moving upwards).

Moving to FIG. 5E, very simple blinking masks 500e are shown with an upward-diagonal-strips pattern. Particularly, a seed mask 500e(s) is defined having alternated blocking areas and passing areas formed by corresponding upward-diagonal strips (extending upwards moving from left to right) with a common width. The other blinking masks 500e are generated from the seed mask 500e(s) by shifting it in a wrap-around manner (extending or reducing the upward-diagonal strips accordingly) along a downward-diagonal direction (extending downwards moving from left to right) by a selected shifting stride. For example, FIG. 5E shows 4 blinking masks 500e each of 12×12 cells. The seed mask 500e(s) has the blocking/passing areas formed by upward-diagonal strips with a width of 2 cells. The other blinking masks 500e are generated from the seed mask 500e(s) by shifting it in a wrap-around manner along the downward-diagonal direction by 1 cell (moving downwards).

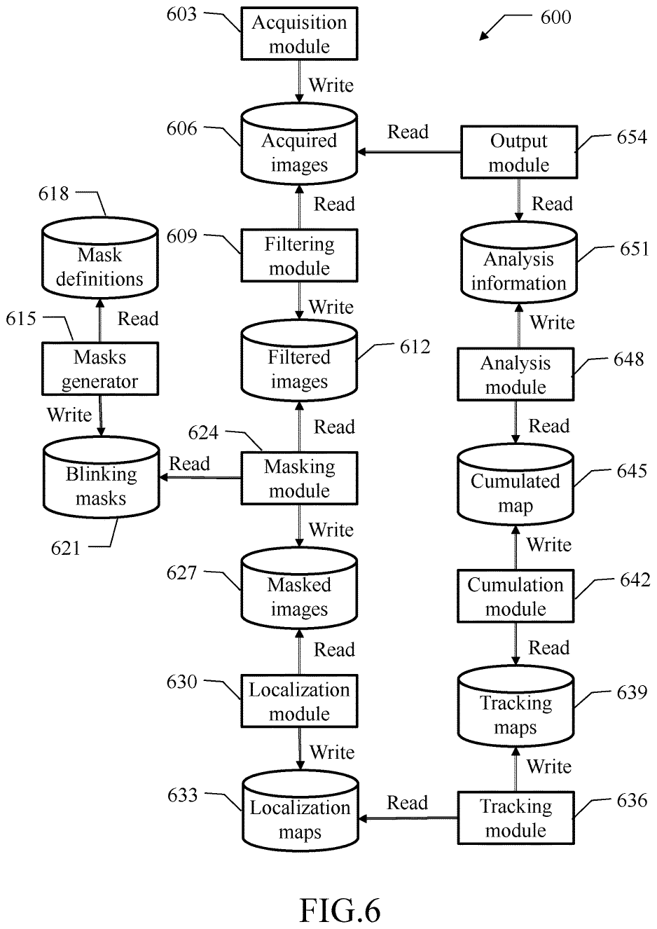

With reference now to FIG. 6, the main software components are shown that may be used to implement the solution according to an embodiment of the present disclosure.

Particularly, all the software components (programs and data) are denoted as a whole with the reference 600. The software components are typically stored in the mass memory and loaded (at least partially) into the working memory of the central unit of the ultrasound scanner when the programs are running, in addition to an operating system and to other application programs not directly relevant to the solution of the present disclosure (thus omitted in the figure for the sake of simplicity). The programs are initially installed into the mass memory, for example, from removable storage units or from the network. In this respect, each program may be a module, segment or portion of code, which comprises one or more executable instructions for implementing the specified logical function.

An acquisition module 603 drives the imaging probe of the ultrasound scanner for acquiring a sequence of acquired images (or frames) of each body-part during an imaging procedure thereof. The acquisition module 603 writes an acquired images repository 606, which contains the acquired images of each imaging procedure that is in progress. The acquired images provide a representation, for example, in standard Brightness mode (B-mode), of a corresponding slice of the body-part (defined by a scan plane of the imaging probe) due to different contributions to the corresponding echo signals of the tissue of the body-part and of the (possible) contrast particles being present therein. Each acquired image is defined by a bitmap comprising a matrix of cells (for example, with 512 rows and 512 columns), with each cell containing a value of a pixel (pixel value), i.e., a basic picture element representing a corresponding location of the body-part having a (physical) size defined by a spatial density of the sensors of the imaging probe; each pixel value defines a brightness of the pixel as a function of an intensity of the corresponding echo signal (for example, from 0 to 256). A filtering module 609 filters the acquired images, especially for removing (or at least substantially reducing) the contribution of the tissue (so as to substantially leave the contribution of the contrast particles only). The filtering module 609 reads the acquired images repository 606 and it writes a filtered images repository 612. The filtered images repository 612 contains a corresponding sequence of filtered images that are generated by filtering the acquired images. Each filtered image is defined by a bitmap comprising a matrix of cells (with the same size as the acquired images), with each cell containing a corresponding pixel value.

In the solution according to an embodiment of the present disclosure, a masks generator 615 may generate the blinking masks at run-time. The masks generator 615 reads a mask definitions repository 618. The mask definitions repository 618 contains a definition of one or more mask sets (of the blinking masks) that may be used to mask the filtered images. The mask definitions repository 618 has an entry for each mask set. The entry stores an indication of a mask pattern of the mask set. Particularly, the mask pattern is defined by its type, i.e., shape and arrangement of the blocking/passing areas of a corresponding seed mask (for example, squares arranged in a chessboard, strips arranged horizontally, vertically, downward-diagonally, upward-diagonally and so on); moreover, the mask pattern is defined by an extent of the blocking/passing areas of the seed mask (or by all possible values thereof), in terms of number of cells (for example, side of squares, width of strips and so on). Moreover, the entry contains information for generating the other blinking masks of the mask set from the seed mask. Particularly, the entry stores an indication of one or more shifting directions (such as horizontal, vertical, downward-diagonal, upward-diagonal and so on). For each shifting direction, the entry stores all the possible shifting strides in terms of number of cells (such as defined by a possible range thereof). The masks generator 615 may also expose a user interface for selecting an accuracy level of the ultrasound scanner (for example, via a virtual slider). In this case, the mask definitions repository 618 also contains (generation) information for generating the blinking masks for all the possible accuracy levels. For example, the mask definitions repository 618 comprises a table that lists the accuracy levels with their generation information. The generation information starts with a preferred mask set with the highest shifting stride for the lowest accuracy level. The shifting stride decreases as the accuracy level increases; once a minimum value (such as 1 cell) of the shifting stride has been reached, a next mask set with the highest shifting stride (in decreasing order of preference) is listed and so on. The masks generator 615 writes a blinking masks repository 621, which contains the blinking masks to be applied to the filtered images. Each blinking mask is defined by a matrix of cells (with the same size as the filtered images), with each cell containing a (binary) masking flag; the masking flag defines whether the cell belongs to a blocking area or to a passing area, for example, asserted (such as at the logic value 1) for the blocking area and deasserted (such as at the logic value 0) for the passing area. A masking module 624 masks the filtered images with the blinking masks. The masking module 624 reads the filtered images repository 612 and the blinking masks repository 621, and it writes a masked images repository 627. The masked images repository 627 contains corresponding masked images that are generated by applying the blinking masks to each filtered image. Each masked image is defined by a bitmap comprising a matrix of cells (with the same size as the filtered images), with each cell storing a corresponding pixel value.

A localization module 630 localizes the (distinguishable) contrast particles that are present in the masked images. The localization module 630 reads the masked images repository 627 and it writes a localization maps repository 633. The localization maps repository 633 contains a sequence of localization maps corresponding to the filtered images, each indicating the positions of the contrast particles that have been localized in all the masked images being generated from the corresponding filtered image. Each localization map is defined by a matrix of cells (with the same size as the masked images), with each cell containing a (binary) localization flag; the localization flag is asserted (such as at the logic value 1) when a contrast particle has been localized at the corresponding cell in one or more masked images and it is deasserted (such as at the logic value 0) otherwise. A tracking module 636 tracks the (positions of the) contrast particles along the localization maps to reconstruct their trajectories. The tracking module 636 reads the localization maps repository 633 and it writes a tracking maps repository 639. The tracking maps repository 639 contains a sequence of tracking maps corresponding to the localization maps, which tracking maps provide an indication of the trajectories of the contrast particles in the body-part. For example, each tracking map is defined by a matrix of cells (with the same size as the localization maps), with each cell containing a tracking value. The tracking value has a null value (for example, 0) if no contrast particle has been localized or a different value if a contrast particle has been localized at the corresponding cell of the localization map, particularly, if the same contrast particle has been localized in the preceding localization map as well, the tracking value contains an indication of the position (defined by coordinates, i.e., row and column, of the corresponding cell) of the (paired) contrast particle in the preceding localization map, whereas otherwise the tracking value contains an appearance flag (for example, −1) indicating that the contrast particle has just appeared. A cumulation module 642 cumulates information relating to the contrast particles derived from the tracking maps. The cumulation module 642 reads the tracking maps repository 639 and it writes a cumulated map repository 645. The cumulated map repository 645 contains a cumulated map defined by a matrix of cells (with the same size as the tracking maps), with each cell containing a cumulated value; the cumulated value is indicative of a characteristic of the contrast particles being localized at the cell, for example, their density and/or speed, or a null value (for example, 0) otherwise. An analysis module 648 generates analysis information of the body-part derived from the trajectories of the contrast particles. Particularly, the analysis information may comprise one or more super-resolution images each providing an indication of the contrast particles along their trajectories (and then of the corresponding blood vessels of the body-part). The super-resolution image is defined by a matrix of cells (with the same size as the cumulated map), with each cell containing a pixel value depending on a characteristic of the corresponding contrast particles, for example, their density or speed. In addition or in alternative, the analysis information may comprise one or more global parameters of the body-part or of a Region of Interest (ROI) thereof, for example, of morphological type (such as blood vessel density, blood vessel tortuosity and so on) and/or hemodynamic type (such as perfusion index, blood volume and so on). The analysis module 648 reads the cumulated map repository 645 and it writes an analysis information repository 651 that stores the analysis information. The analysis module 648 also exposes a user interface for selecting the type of analysis information to be provided. An output module 654 outputs the analysis information (for example, on the monitor of the ultrasound scanner), either alone or in association with the acquired images. For this purpose, the output module 654 reads the analysis information repository 651 and possibly the acquired images repository 606.

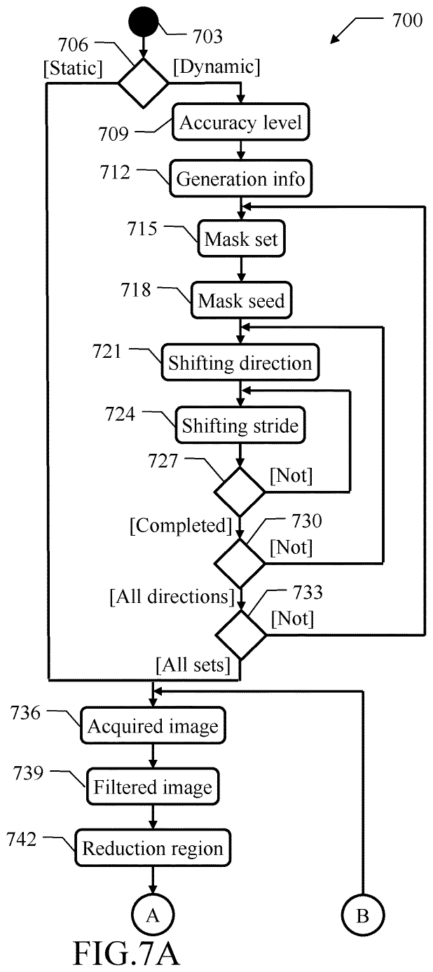

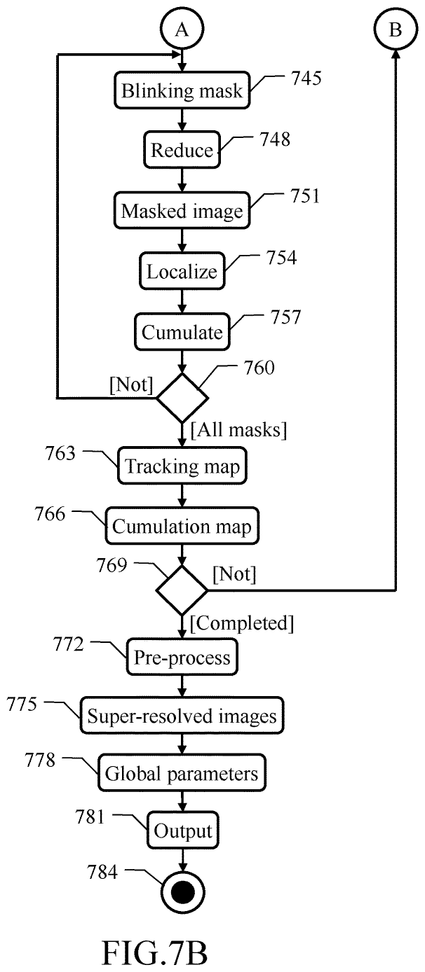

With reference now to FIG. 7A-FIG. 7B, an activity diagram is shown describing the flow of activities relating to an implementation of the solution according to an embodiment of the present disclosure.

Particularly, the diagram represents an exemplary process that may be used to image the body-part of a patient with a method 700. In this respect, each block may correspond to one or more executable instructions for implementing the specified logical function on the central unit of the ultrasound scanner.

Before a corresponding imaging procedure is started, a healthcare operator (for example, a nurse) administers the contrast agent to the patient. For example, the contrast agent is a suspension of gas-filled bubbles in a liquid carrier. The gas-filled bubbles are generally stabilized by entraining or encapsulating the gas or a precursor thereof into a variety of systems, comprising phospholipids, emulsifiers, oils, thickeners, sugars, proteins or polymers; stabilized gas-filled bubbles are generally referred to as microvesicles. Particularly, microvesicles dispersed in an aqueous medium and bounded at the gas/liquid interface by a very thin envelope involving a surfactant (i.e., an amphiphilic material) are also known as microbubbles; alternatively, microvesicles surrounded by a solid material envelope formed by lipids or (natural or synthetic) polymers are also known as microballoons or microcapsules. Another kind of contrast agent comprises a suspension of porous microparticles of polymers or other solids, which carry bubbles of gas entrapped within the pores of the microparticles, or adsorbed on their surfaces. An example of a commercial contrast agent comprising microvesicles is Sono Vue by Bracco International BV (trademarks). For example, the contrast agent is administered to the patient intravenously as a bolus (i.e., a single dose provided with a syringe over a short period of time, of the order of 2-20 s); as a consequence, the contrast agent circulates within a circulatory system of the patient, so as to perfuse the body-part.

The physician then switches on the ultrasound scanner and places its imaging probe in contact with a skin of the patient in the area of the body-part. In response thereto, the process begins by passing from black start circle 703 to block 706. The flow of activity now branches according to a configuration of the ultrasound scanner (for example, set manually, defined by default or the only one available). Particularly, if the blinking masks are to be generated dynamically, the masks generator at block 709 obtains the accuracy level. For example, the accuracy level may be entered manually by the physician (via the user interface of the masks generator) or it may be read from a configuration parameter (customizable starting from a default value). In response thereto, the masks generator at block 712 retrieves the generation information corresponding to the accuracy level from the mask definitions repository (as defined by one or more mask sets each with its mask pattern, shifting directions and corresponding shifting strides). The masks generator at block 715 takes into account a (current) mask set indicated in the generation information (starting from a first one in decreasing order of preference). The masks generator at block 718 builds the seed mask of the mask set (saving it into the corresponding repository) according to its mask pattern as defined by the type, i.e., shape and arrangement, of the blocking/passing areas and by their extent. The masks generator at block 721 takes into account a (current) shifting direction of the mask set (starting from a first one in any arbitrary order). The masks generator at block 724 builds another blinking mask of the mask set (saving it into the corresponding repository) by shifting a previously built blinking mask of the mask set (starting from the seed mask) along the shifting direction by the shifting stride. The masks generator at block 727 verifies whether this blinking mask is the same as the seed mask (meaning that all the blinking masks of the mask set for the shifting direction have been generated). If not, the process returns to block 724 to repeat the same operations. Conversely (once the generation of the mask set has been completed), the flow of activity descends into block 730, wherein the masks generator verifies whether all the shifting directions of the mask set have been applied. If not, the process returns to block 721 to repeat the same operations for a next shifting direction. Conversely (once all the shifting directions have been applied), the flow of activity descends into block 733, wherein the masks generator verifies whether all the mask sets have been generated. If not, the flow of activity returns to block 715 to repeat the same operations for a next mask set. Conversely (once all the mask sets have been generated), the flow of activity descends into block 736; the same point is also reached directly from block 706 if the accuracy level has not changed from a previous imaging procedure (so that the corresponding blinking masks have already been generated) or if the blinking masks are defined statically in the corresponding repository (without the mask definitions repository and the masks generator).

The acquisition module now continually acquires (for example, with an acquisition rate of 500-5,000 images per second) acquired images of its field of view comprising the body-part (saving them into the corresponding repository). Particularly, the acquisition module drives the imaging probe at each acquisition instant to apply a sequence of ultrasound waves with low acoustic energy (for example, with low mechanical index MI=0.01-0.4, so as to involve a negligible destruction of the contrast agent) and to record the echo signals being received in response thereto (for example, in the form of Radio-Frequency (RF) or In-phase Quadrature (IQ) signals); the echo signals are processed (for example, pre-amplified, digitalized, beam formed, video converted, log-compressed, scan converted and so on) so as to create the corresponding acquired image. The acquisition module may work in a contrast-specific imaging mode so as to substantially remove, or at least reduce, the dominant (linear) contribution of the tissue in the echo signals with respect to the (non-linear) contribution of the contrast agent (for example, with harmonic imaging (HI), pulse inversion (PI), power modulation (PM) or contrast pulse sequencing (CPS) techniques). The filtering module at block 739 filters each (current) acquired image (extracted from the corresponding repository) into its filtered image (saving it into the corresponding repository). Particularly, the filtering module may pre-process the acquired image by applying motion correction techniques, for example, image-intensity based (as so to bring the acquired image into spatial correspondence with a reference image, such as a first acquired image). In any case, the filtering module filters the acquired image to remove, or at least substantially reduce, the contribution of the tissue thereto; for example, the filtering module may apply a spatiotemporal filtering technique (such as based on Singular Value Decomposition, SVD) that exploits a spatiotemporal coherence of a motion of the tissue and of the contrast particles (flowing with the blood) to discriminate them. The filtering module may also post-process the filtered image to denoise it, for example, by applying intensity-thresholding techniques.

In the solution according to an embodiment of the present disclosure, the masking module at block 742 determines a reduction region of the filtered image wherein the contrast particles are present. For example, the reduction region is set to the biggest rectangle containing all the contrast particles. The reduction region may be represented by a reduction mask, which is defined by a matrix of cells (with the same size as the filtered images), with each cell containing a (binary) reduction flag, which is deasserted (such as at the logic value 0) when the corresponding pixel of the filtered image belongs to the reduction region and it is asserted (such as at the logic value 1) otherwise. The masking module at block 745 retrieves a (current) blinking mask from the corresponding repository (starting from a first one in their storing order). The masking module at block 748 generates a reduced version of the blinking mask according to the reduction region. For example, the masking module sums the reduction mask and the blinking mask pixel-by-pixel, so as to maintain the blinking mask unaltered in the reduction region whereas all its masking flags are forced to be asserted outside the reduction region (so that the whole area outside the reduction region becomes a new blocking area). The masking module at block 751 applies the (reduced) blinking mask to the filtered image (just added to the corresponding repository) so as to generate its masked image (saving it into the corresponding repository). For this purpose, the masking module multiplies the filtered image by the blinking mask pixel-by-pixel, so as to maintain the pixel values of the filtered image in the passing areas of the blinking mask (within the reduction region), whereas all the other pixel values (in the blocking areas of the blinking mask within the reduction region and always outside the reduction region) are reset to a null value (0). The localization module at block 754 localizes any contrast particles that are distinguishable in the masked image (just added to the corresponding repository) within the reduction region. Particularly, the localization module detects the (distinguishable) contrast particles, for example, by applying a PSF cross-correlation technique (searching for structures exhibiting a good correlation to the PSF of the contrast particles). The localization module then determines the positions of these contrast particles, for example, their centroids by applying a peak detection technique. The localization module at block 757 cumulates the (positions of the) contrast particles being localized into the localization map associated with the filtered image (in the corresponding repository). Particularly, for each contrast particle being localized the localization module asserts the localization flag of the corresponding cell in the localization map (initialized with all the localization flags being deasserted). In this phase, it may happen that same contrast particles are each localized multiple times in different masked images at slightly different positions (because of the corresponding different masking of the contrast particle and of any other overlapped contrast particles). These multiple localizations of the contrast particles do not influence the obtained information about the morphology of the body-part, but they may lead to an overestimate of its vascular density (depending on the number of contrast particles being localized). Therefore, if necessary, it is possible to filter the multiple localizations of the contrast particles, for example, by combining the contrast particles that are spaced apart by less than a (differentiation) threshold (such as corresponding to 0.3-0.7 times the size of the contrast particles, like 2-3 pixels) into a single contrast particle at a position in the middle of them. The masking module at block 760 verifies whether all the blinking masks have been applied to the filtered image. If not, the process returns to block 745 to repeat the same operations with a next blinking mask. Conversely (once all the blinking masks have been applied), the flow of activity descends into block 763. The tracking module now generates the tracking map associated with the filtered image (saving it into the corresponding repository) by tracking the contrast particles of the current localization map (just added to the corresponding repository) with respect to the contrast particles of a preceding localization map (previously added to the corresponding repository), if any, to reconstruct their trajectories. For example, the tracking module attempts to pair each contrast particle of the current localization map with a corresponding contrast particle of the preceding localization map (such as by applying minimal-distance techniques). For each contrast particle of the current localization map, if it has been paired the tracking module sets the corresponding tracking value to the coordinates of the corresponding contrast particle in the preceding localization map, whereas otherwise (always true for a first localization map) the tracking module sets the corresponding tracking value to the appearance flag; in any case, the tracking module adds the tracking value to the corresponding cell in the tracking map (initialized with all the tracking values at the null value). The cumulation module at block 766 updates the cumulation map (in the corresponding repository, initialized with all the cumulated values at the null value) by cumulating the information relating to the contrast particles being derived from the tracking maps (in the corresponding repository). For example, the cumulation module determines any (paired) set defined by the contrast particles that are paired along a minimum number (such as 2-10) of consecutive tracking maps, thereby indicating the positions of a same contrast particle along them. Particularly, when the minimum number is higher than 2, this allows rejecting contrast particles with corresponding lower persistence. The cumulation module then updates the cumulated values corresponding to the positions of the contrast particle of each paired set accordingly; for example, when the cumulated values indicate the density of the contrast particles, the cumulation module increments each of them by 1, whereas when the cumulated values indicate the speed of the contrast particles, the cumulation module calculates the speed of the contrast particle at each position thereof (as a distance from the position of the contrast particle in the corresponding tracking map to the position of the contrast particle in the previous tracking map divided by a time elapsed between them, given by an inverse of the acquisition rate) and sets the corresponding cumulated value to it (when at the null value) or to an average with its previous value otherwise. The cumulation module at block 769 verifies whether the cumulation map has been completed. For example, this happens in response to any stop condition, such as when no new distinguishable contrast particle is localized in one or more consecutive filtered images (such as 1-10), after a pre-defined time from a beginning of the imaging procedure (such as 10-30 s) or in response to a command entered on the ultrasound scanner. If not, the process returns to block 736 to repeat the same operations on a next acquired image. Conversely (once the cumulated map has been completed), the flow of activity descends into block 772.

At this point, the analysis module may pre-process the cumulated map (in the corresponding repository), for example, filling discontinuities by applying spatial low-pass filtering techniques. Continuing to block 775, if the analysis information comprises one or more super-resolution images (for example, selected manually, defined by default or the only ones available), the analysis module generates each of them (saving it into the corresponding repository) from the (possibly pre-processed) cumulated map (extracted from the corresponding repository). For example, each super-resolution image is generated by rendering the corresponding cumulated values (i.e., density or speed) according to a given color-map palette (with brightness increasing with the cumulated values). Continuing to block 778, if the analysis information comprises one or more global parameters in addition or in alternative to the super-resolution images (for example, again selected manually, defined by default or the only ones available), the analysis module calculates each of them (saving it into the corresponding repository) from the (possibly pre-processed) cumulated map (extracted from the corresponding repository). For example, the analysis module prompts the physician to select a desired ROI in any (acquired/super-resolution) image of the body-part (up to it entirely); the analysis module then calculates the global parameter from the corresponding cumulated values (i.e., density or speed), for example, by averaging, integrating and so on. The output module at block 781 outputs the analysis information (retrieved from the corresponding repository). The output information may be provided in different modes (for example, selected manually, defined by default or the only one available). For example, the super-resolution images and/or the global parameters are displayed alone or side-by-side with the acquired images, each super-resolution image is displayed superimposed on the acquired images, and so on. The above-mentioned operations may be reiterated one or more times by selecting different types of analysis information and/or different accuracy levels. For example, it is possible to select a (relatively) low accuracy level to be used during the imaging procedure, so as to obtain the analysis information substantially in real-time; if necessary, it is then possible to increase the accuracy level for use on the acquired images extracted from the corresponding repository after completing the imaging procedure, so as to allow investigating the analysis information more deeply off-line. Once the process has been completed (as indicated by a corresponding command entered on the ultrasound scanner), the flow of activity ends to the concentric white/black stop circles 784.

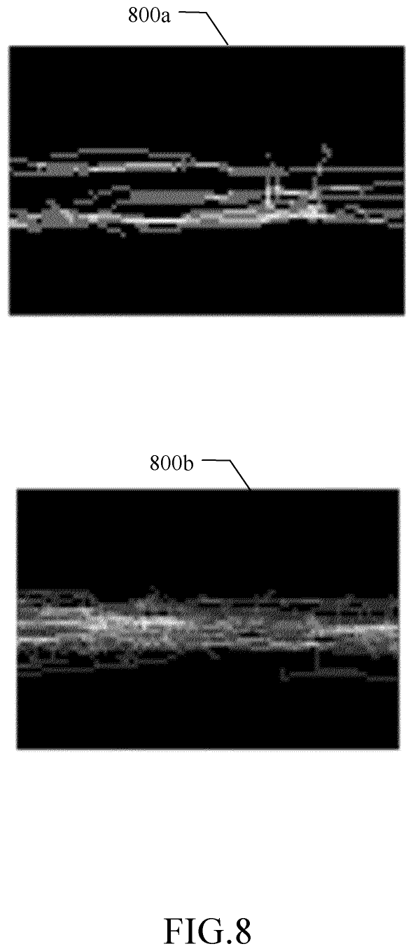

With reference now to FIG. 8, a comparative example is shown of in-vitro imaging procedures known in the art and according to an embodiment of the present disclosure.

Particularly, a sequence of acquired images have been acquired of a microflow phantom with a high concentration of a contrast agent over a short acquisition time of a few seconds. The sequence of acquired images has been processed with a conventional UML technique to obtain a corresponding super-resolution image 800a and with the above-described technique according to an embodiment of the present disclosure to obtain another corresponding super-resolution image 800b (both of them showing the speed of the contrast particles that have been localized with corresponding pixel values having brightness increasing with the speed).

As can be seen, the super-resolution image 800b shows more details of the microflow phantom than the super-resolution image 800a does (despite the high concentration of the contrast agent and the short acquisition time).

### Modifications

In order to satisfy local and specific requirements, a person skilled in the art may apply many logical and/or physical modifications and alterations to the present disclosure, provided that it remains within the scope of the claims. More specifically, although this disclosure has been described with a certain degree of particularity with reference to one or more embodiments thereof, it should be understood that various omissions, substitutions and changes in the form and details as well as other embodiments are possible. Particularly, different embodiments of the present disclosure may be practiced even without the specific details (such as the numerical values) set forth in the preceding description to provide a more thorough understanding thereof; conversely, well-known features may have been omitted or simplified in order not to obscure the description with unnecessary particulars. Moreover, it is expressly intended that specific elements and/or method steps described in connection with any embodiment of the present disclosure may be incorporated in any other embodiment as a matter of general design choice. Moreover, items presented in a same group and different embodiments, examples or alternatives are not to be construed as de facto equivalent to each other (but they are separate and autonomous entities). In any case, each numerical value should be read as modified according to applicable tolerances; particularly, unless otherwise indicated, the terms “substantially”, “about”, “approximately” and the like should be understood as within 10%, preferably 5% and still more preferably 1%. Moreover, each range of numerical values should be intended as expressly specifying any possible number along the continuum within the range (comprising its end points). Ordinal or other qualifiers are merely used as labels to distinguish elements with the same name but do not by themselves connote any priority, precedence or order. The terms include, comprise, have, contain, involve and the like should be intended with an open, non-exhaustive meaning (i.e., not limited to the recited items), the terms based on, dependent on, according to, function of and the like should be intended as a non-exclusive relationship (i.e., with possible further variables involved), the term a/an should be intended as one or more items (unless expressly indicated otherwise), and the term means for (or any means-plus-function formulation) should be intended as any structure adapted or configured for carrying out the relevant function.

For example, an embodiment provides an imaging method of ultrasound type for imaging a body-part of a patient. However, the body-part may be of any type (for example, organs, regions thereof, tissues, bones, joints and so on) and in any condition (for example, healthy, pathological with any lesions and so on), and it may belong to any patient (for example, human beings, animals and so on); moreover, the imaging method may be of any ultrasound type (for example, different frequency of the ultrasound waves, gain, time-gain compensation and so on) and used in any medical imaging application (for example, diagnostic, therapeutic, surgical and so on). In any case, although the imaging method may facilitate the task of a physician, it only provides intermediate results that may help him/her but with the medical activity stricto sensu that is always made by the physician himself/herself.

In an embodiment, the imaging method comprises the following steps under the control of a computing system. However, the computing system may be of any type (see below).

In an embodiment, the imaging method comprises receiving (by the computing system) a sequence of input images. However, the input images may be received in any way (for example, in real-time/off-line, acquired, downloaded from a network, read from a storage device, in any number, with any frequency, and so on) and they may be of any type (for example, with any size, chromaticity, bit depth, real/complex values and so on).

In an embodiment, the input images are representative of any contrast particles of a contrast agent that are comprised in the body-part. However, the contrast particles may be of any type (for example, gas-filled bubbles, phase-change nanodroplets and so on) and each input image may represent any number of contrast particles in any way (for example, with pixel/voxel values, in positive/negative form, each one with any extent and so on). The contrast agent may have been administered to the patient in any way (for example, as a bolus by a syringe, as a continuous infusion by a pump and so on) and at any time (for example, in advance, immediately before performing the imaging method, continuously during it and so on); the contrast agent may also be administered to the patient in a non-invasive manner (for example, orally for imaging the gastro-intestinal tract or via a nebulizer into the airways) or without any substantial physical intervention on the patient that would require professional medical expertise or entail any health risk (for example, intramuscularly). Moreover, the application to an endogenous contrast agent (requiring no administration thereof to the patient) is not excluded. In any case, this is a computer-implemented method only comprising steps performed by the computing system (which steps may be performed even independently of the acquisition of the input images and then without requiring any interaction with the patient.

In an embodiment, the imaging method comprises generating (by the computing system) a plurality of masked images from each of at least part of the input images by applying corresponding blinking masks thereto. However, the blinking masks may be in any number and provided in any way (for example, generated, read, downloaded and so on); moreover, the blinking masks may be applied to the input images in any way (for example, to all the input images, to a sub-set thereof obtained by temporal sub-sampling, until any stop condition is satisfied, to the whole of each input image, to a portion thereof and so on).

In an embodiment, each of the blinking masks alternates a plurality of blocking areas and a plurality of passing areas wherein the input image is masked and not masked, respectively. However, the blocking/passing areas may be in any number, with any pattern (for example, regular, irregular and so on) and of any extent (for example, either the same or different between them); for example, the blocking/passing areas may be in the form of a chessboard, alternated strips having any direction, stars, perpendicular rectangles, “+”-like areas, “X”-like areas, “T”-like areas, circles, ovals and so on.

In an embodiment, the imaging method comprises determining (by the computing system) corresponding positions of any distinguishable ones of the contrast particles being distinguishable from any other ones of the contrast particles in each of the masked images. However, the distinguishable contrast particles may be in any number in each masked image, and their positions may be determined in any way (for example, detecting the distinguishable contrast particles with PSF cross-correlation, deconvolution, entropy and the like techniques and determining their positions with peak detection, weighted average, curve fitting and the like techniques, localizing the contrast particles only in the masked images or in addition to their preliminary localization in the input images being not masked, distinguishing the contrast particles that are overlapped to any degree, filtering or not multiple detections of a same contrast particle, and so on).

In an embodiment, the imaging method comprises outputting (by the computing system) analysis information of the body-part. However, the analysis information may be output in any way (for example, substantially simultaneously with the receipt of the input images or with any delay from it, as a post-processing of the input images, displayed on a monitor or on virtual-reality glasses, printed, transmitted remotely and so on).

In an embodiment, the analysis information is provided on the basis of the positions of the distinguishable contrast particles. However, the analysis information may be of any type (for example, one or more super-resolution images, global parameters, histograms, video-clips and so on) and based on the positions of the distinguishable contrast particles in any way (for example, derived from their number, movement, presence and so on).

Further embodiments provide additional advantageous features, which may however be omitted at all in a basic implementation. In this respect, it is expressly intended that the features of each of the following embodiments may be combined with the above features either alone or in combination with the features of any number of the other following embodiments.

In an embodiment, the passing areas of all the blinking masks do not mask a whole analysis region of the input images. However, this result may be achieved in any way (for example, with the passing areas of the different blinking masks that are disjoint, at least in part overlapped and so on) for any analysis region of the input images (for example, equal to the whole extent of the input images, to a portion thereof determined manually or automatically to be of interest for the medical imaging application, and so on).

In an embodiment, the imaging method comprises receiving (by the computing system) a sequence of acquired images. However, the acquired images may be received in any way (for example, either the same or different with respect to the input images) and they may be of any type (for example, either in the same number or not and of the same type or not with respect to the input images, and so on).

In an embodiment, the acquired images are images that have been acquired of the body-part. However, the acquired images may have been acquired in any way (for example, with different frequencies, mechanical indexes and so on).

In an embodiment, the imaging method comprises generating (by the computing system) the input images from the acquired images by filtering a contribution of a tissue of the body-part thereto. However, the acquired images may be filtered in any way (for example, by applying spatiotemporal, nonlocal means, high-pass or thresholding techniques, with or without any pre-processing and/or post-processing, such as motion correction, denoising and so on) to reduce the contribution of the tissue to any extent; in any case, the possibility is not excluded of receiving the acquired images already filtered (for example, when they have been acquired in contrast-specific mode).

In an embodiment, the imaging method comprises tracking (by the computing system) the positions of the distinguishable contrast particles being determined in the masked images of each of the input images for reconstructing one or more trajectories thereof in the body-part. However, the distinguishable contrast particles may be tracked in any way (for example, by applying minimal-distance, Markov chain, cross-correlation, assignment and so on techniques, with or without a persistence control of any length, and so on).

In an embodiment, the imaging method comprises generating (by the computing system) the analysis information according to the trajectories of the distinguishable contrast particles. However, the analysis information may be generated according to the trajectories of the distinguishable contrast particles in any way (for example, based on density, speed and the like of the distinguishable contrast particles, with or without the application of any processing for reducing discontinuities, such as based on average, interpolation, sparsity-promoting and the like technique, and so on).

In an embodiment, the imaging method comprises generating (by the computing system) the analysis information comprising at least one super-resolution image indicative of the positions and the trajectories of the distinguishable contrast particles. However, the super-resolution images may be in any number and of any type (for example, in color or black-and-white, with the distinguishable contrast particles represented along their trajectories by any markers being smaller than the resolution-limit, such as single pixels corresponding to the positions of the distinguishable contrast particles, spots around their positions with an extent corresponding to a size of the contrast particles and so on), and they may be output in any way (for example, alone, together with or superimposed on the acquired images, and so on).

In an embodiment, the imaging method comprises generating (by the computing system) the analysis information comprising at least one global parameter based on the trajectories of the distinguishable contrast particles. However, the global parameters may be in any number and of any type (for example, partial, different and additional global parameters with respect to the ones mentioned above) and they may be output in any way (for example, in numerical form, graphical form, alone or together with the acquired images, and so on).

In an embodiment, each of the passing areas has an extent with at least one dimension equal to 0.5-1.5 times an extent of a representation of each of the contrast particles in the input images. However, the extent of the representation of each contrast particle may be determined in any way (for example, analytically, experimentally and so on) and the passing areas may be defined accordingly in any way (for example, setting any number of dimensions thereof to any multiple of it, either the same or different among them, and so on).

In an embodiment, each of the passing areas has an extent with at least one dimension equal to 0.25-0.75 times a wavelength of an ultrasound signal being used to obtain the input images. However, the possibility is not excluded of setting the passing areas in any other way according to the wavelength being used to obtain (directly or indirectly) the input images, or even independently of it.

In an embodiment, the blinking masks comprise one or more mask sets for corresponding mask patterns. However, the mask sets may be in any number for any mask patterns (for example, chessboard, strips, grid and so on); for example, it is possible to have a single mask set for the chessboard pattern, a single mask set for one of the strips patterns (corresponding to a prevalent motion direction of the contrast particles), two mask sets for the horizontal/vertical strips pattern, two mask sets for the downward-diagonal/upward-diagonal strips pattern, any combination thereof and so on.

In an embodiment, each of the mask sets comprises a plurality of the blinking masks having the blocking areas and the passing areas thereof featuring the mask pattern of the mask set with corresponding displacements along one or more shifting directions. However, the displacements may be of any type (for example, along any number of shifting directions, with any shifting stride and so on).

In an embodiment, the mask patterns comprise a chessboard pattern with the blocking areas and the passing areas of rectangular shape being alternated along two perpendicular directions. However, the chessboard pattern may be of any type (for example, with (square/not-square) rectangles of any extent, with any orientation and so on).

In an embodiment, the mask patterns comprise at least one strips pattern with the blocking areas and the passing areas having a strip shape extending along a common direction. However, the strips pattern may be of any type (for example, with strips of any extent, either the same or different between the blocking areas and the passing areas, and so on).

In an embodiment, the blinking masks are defined by corresponding matrices of cells each having a plurality of rows and a plurality of columns. However, the blinking masks may have any number of rows and columns, with the cells corresponding to locations of the body-part of any type (for example, 2D/3D, with any size, density and so on).

In an embodiment, the common direction of the blocking areas and of the passing areas extend along the rows, the columns, a first diagonal and/or a second diagonal of the matrices. However, the common direction of the blocking/passing areas may be of any type (for example, partial, different and additional directions with respect to the ones mentioned above, such as extending obliquely to the rows/columns with any angle, and so on).

In an embodiment, the imaging method comprises (for each of the mask sets) providing (by the computing system) an indication of the shifting directions and an indication of a shifting stride and of a shifting number for each of the shifting directions. However, the shifting directions may be in any number and of any type (for example, leftwards, rightwards, downwards, upwards, obliquely with any direction and angle, and so on) and each of them may have any shifting stride and shifting number (for example, either the same or different among the shifting directions), with this information that may be provided in any way (for example, retrieved according to a selected accuracy level, entered directly, pre-defined, with the shifting number that is received or calculated according to the mask pattern, and so on).

In an embodiment, the imaging method comprises (for each of the mask sets) providing (by the computing system) a seed mask of the blinking masks of the mask set. However, the seed mask may be any blinking mask of the mask set and it may be provided in any way (for example, built according to a definition thereof, retrieved from a stored representation thereof, and so on).

In an embodiment, the imaging method comprises (for each of the mask sets) generating (by the computing system) the other blinking masks of the mask set by shifting the seed mask along each of the shifting directions by the shifting stride for the shifting number of the shifting direction. However, this operation may be performed at any time (for example, at run-time for generating the blinking masks dynamically for each imaging procedure, in advance for pre-defining the blinking masks and so on) and in any way (for example, by simply shifting in a wrap-around manner, extending and/or reducing the passing/blocking areas accordingly and so on); in any case, the possibility is not excluded of generating the blinking masks in a different way (for example, randomly).

In an embodiment, the imaging method comprises receiving (by the computing system) an indication of an accuracy level for imaging the body-part. However, the accuracy level may be of any type (for example, discrete/continuous, qualitative/quantitative, with any number of possible values or range of values, and so on) and it may be received in any way (for example, entered via any software/hardware input unit, such as a slider, up/down buttons, a knob, an input box and the like, read from any storage structure and so on).

In an embodiment, the imaging method comprises selecting (by the computing system) the mask sets according to the accuracy level. However, the mask sets may be selected in any way (for example, via a table, a formula, a rule and so on) and among any number and type of available mask sets.

In an embodiment, the imaging method comprises selecting (by the computing system) an extent of the blocking areas and of the passing areas of the blinking masks of each of the mask sets according to the accuracy level. However, the extent of the blocking/passing areas may be selected in any way (for example, either the same or different with respect to above) and among any values (for example, in any range, with any pitch and so on).

In an embodiment, the imaging method comprises selecting (by the computing system) the shifting directions of each mask set according to the accuracy level. However, the shifting directions may be selected in any way (for example, either the same or different with respect to above) and among any number and type of available shifting directions.

In an embodiment, the imaging method comprises selecting (by the computing system) the shifting stride for each of the shifting directions of each mask set according to the accuracy level. However, the shifting stride may be selected in any way (for example, either the same or different with respect to above) and among any values (for example, in any range, with any pitch and so on).

In an embodiment, the imaging method comprises (for each of the input images) determining (by the computing system) a reduction region of the input image wherein the corresponding contrast particles are represented. However, the reduction region may be determined in any way (for example, the biggest rectangle containing all the contrast particles, any percentage thereof, with or without exclusion of any singular contrast particles whose distance from the other contrast particles is higher than any threshold, and so on).

In an embodiment, the imaging method comprises (for each of the input images) setting (by the computing system) an application of the blinking masks to the input image according to the reduction region thereof. However, the application of the blinking masks may be set according to the reduction region in any way (for example, by forcing the portion of the blinking masks outside the reduction region to mask the input images so as to always disregard the corresponding portion of the input image, by reducing the size of the blinking masks according to the reduction region, directly or via the seed masks enlarged up to make them regular, and applying these blinking masks only to the reduction region of the input image, and so on); in any case, the possibility is not excluded of always applying the blinking mask indiscriminately to the whole input images.

In an embodiment, the imaging method comprises filtering (by the computing system) any multiple determinations of different positions of each of the distinguishable contrast particles in the masked images of each of the input images. However, the multiple determinations of the same distinguishable contrast particles may be filtered in any way (for example, incrementally/cumulatively, according to any minimum distance among them, with spatial filtering techniques and so on).

Generally, similar considerations apply if the same solution is implemented with an equivalent method, provided that it remains within the scope of the claims (by using similar steps with the same functions of more steps or portions thereof, removing some steps being non-essential, or adding further optional steps); moreover, the steps may be performed in a different order, concurrently or in an interleaved way (at least in part).

An embodiment provides a computer program, which is configured for causing a computing system to perform the above-mentioned imaging method when the computer program is executed on the computing system. An embodiment provides a computer program product, which comprises a computer readable storage medium embodying a computer program, the computer program being loadable into a working memory of a computing system thereby configuring the computing system to perform the same imaging method. However, the (computer) program may be of any type (for example, implemented as a stand-alone module, a plug-in for a pre-existing application, such as a manager of the ultrasound scanner, directly in the latter and so on) and it may be used on any computing system (see below).

Generally, similar considerations apply if the program is structured in a different way, or if additional modules or functions are provided; likewise, the memory structures may be of other types, or they may be replaced with equivalent entities (not necessarily consisting of physical storage media). The program may take any form suitable to be used by the computing system, thereby configuring it to perform the desired operations; particularly, the program may be in the form of external or resident software, firmware, or microcode (either in object code or in source code), for example, to be compiled or interpreted. Moreover, it is possible to provide the program on any computer readable storage medium. The storage medium is any tangible medium (different from transitory signals per se) that may retain and store instructions for use by the computing system. For example, the storage medium may be of electronic, magnetic, optical, electromagnetic, infrared, or semiconductor type; examples of such storage medium are fixed disks (where the program may be pre-loaded), removable disks, memory keys (for example, USB), and the like. The program may be downloaded to the computing system from the storage medium or via a network (for example, the Internet, a wide area network and/or a local area network comprising transmission cables, optical fibers, wireless connections, network devices); one or more network adapters in the computing system receive the program from the network and forward it for storage into one or more storage devices of the computing system. In any case, the solution according to an embodiment of the present disclosure lends itself to be implemented even with a hardware structure (for example, by electronic circuits integrated on one or more chips of semiconductor material), or with a combination of software and hardware suitably programmed or otherwise configured.

An embodiment provides a computing system, which comprises means configured for performing the steps of the above-mentioned imaging method. An embodiment provides a computing system comprising corresponding circuitries (i.e., any hardware suitably configured, for example, by software) for performing the steps of the same imaging method. However, the computing system may be of any type (for example, a central unit of an imaging system, a separate computer and so on).

An embodiment provides an imaging system of ultrasound type comprising the above-mentioned computing system. However, the imaging system may be of any type (for example, a scanner, an endoscope, a laparoscope and so on).

In an embodiment, the imaging system comprises an imaging probe for acquiring the sequence of input images. However, the imaging probe may be of any type (for example, hand-held, fixed, of matrix or liner type, and so on).

In an embodiment, the computing system is coupled with the imaging probe for receiving the sequence of input images therefrom. However, computing system and the scanner may be coupled in any way (for example, locally/remotely via any type of wired and/or wireless connection, and so on).

Generally, similar considerations apply if the computing system and the imaging system each has a different structure or comprises equivalent components or has other operative characteristics, provided that it remains within the scope of the claims. In any case, every component thereof may be separated into more elements, or two or more components may be combined together into a single element; moreover, each component may be replicated to support the execution of the corresponding operations in parallel. Moreover, unless specified otherwise, any interaction between different components generally does not need to be continuous, and it may be either direct or indirect through one or more intermediaries.

An embodiment provides a medical method for imaging a body-part of a patient. However, the medical method may be used to image any body-part of any patient (see above).

In an embodiment, the medical method comprises administering a contrast agent to the patient. However, the contrast agent may be of any type and it may be administered in any way (see above).

In an embodiment, the medical method comprises acquiring a sequence of input images representative of any contrast particles of the contrast agent that are comprised in the body-part. However, the input images may be acquired in any way (see above).

In an embodiment, the medical method comprises imaging the body-part according to the above-mentioned imaging method thereby outputting the analysis information. However, the analysis information may be of any type and outputted in any way (see above).

In an embodiment, the medical method comprises performing a medical procedure relating to the body-part according to the analysis information. However, the medical procedure may be of any type (for example, a diagnostic procedure, a therapeutic procedure, a surgical procedure and so on).

In an embodiment, the medical method is a diagnostic method comprising evaluating a health condition of the body-part according to the analysis information. However, the proposed solution may find application in any kind of diagnostic method in the broadest meaning of the term (for example, aimed at discovering new lesions, monitoring known lesions and so on).

In an embodiment, the medical method is a therapeutic method comprising treating the body-part according to the analysis information. However, the proposed solution may find application in any kind of therapeutic method in the broadest meaning of the term (for example, aimed at curing a pathological condition, avoiding its progress, preventing the occurrence of a pathological condition, ameliorating a comfort of the patient and so on).

In an embodiment, the medical method is a surgical method comprising operating the body-part according to the analysis information. However, the proposed solution may find application in any kind of surgical method in the broadest meaning of the term (for example, for curative purposes, prevention purposes, aesthetic purposes and so on).

1. An imaging method of ultrasound type for imaging a body-part of a patient, wherein the imaging method comprises, under the control of a computing system:
receiving, by the computing system a sequence of input images representative of any contrast particles of a contrast agent that are comprised in the body-part,
generating, by the computing system, a plurality of masked images from each of at least part of the input images by applying corresponding blinking masks thereto, each of the blinking masks alternating a plurality of blocking areas and a plurality of passing areas wherein the input image is masked and not masked, respectively,
determining, by the computing system, corresponding positions of any distinguishable ones of the contrast particles being distinguishable from any other ones of the contrast particles in each of the masked images, and
outputting, by the computing system, analysis information of the body-part on the basis of the positions of the distinguishable contrast particles.

2. The imaging method according to claim 1, wherein the passing areas of all the blinking masks not mask a whole analysis region of the input images.

3. The imaging method according to claim 1, wherein the imaging method comprises:
receiving, by the computing system a sequence of acquired images being acquired of the body-part, and
generating, by the computing system the input images from the acquired images by filtering a contribution of a tissue of the body-part thereto.

4. The imaging method according to claim 1, wherein the imaging method comprises:
tracking by the computing system the positions of the distinguishable contrast particles being determined in the masked images of each of the input images for reconstructing one or more trajectories thereof in the body-part, and
generating, by the computing system the analysis information according to the trajectories of the distinguishable contrast particles.

5. The imaging method according to claim 4, wherein the imaging method comprises:
generating by the computing system the analysis information comprising at least one super-resolution image indicative of the positions and the trajectories of the distinguishable contrast particles and/or at least one global parameter based on the trajectories of the distinguishable contrast particles.

6. The imaging method according to claim 1, wherein each of the passing areas has an extent with at least one dimension equal to 0.5-1.5 times an extent of a representation of each of the contrast particles in the input images.

7. The imaging method according to claim 1, wherein each of the passing areas has an extent equal to 0.25-0.75 times a wavelength of an ultrasound signal being used to obtain the input images.

8. The imaging method according to claim 1, wherein the blinking masks comprise one or more mask sets for corresponding mask patterns, each of the mask sets comprising a plurality of the blinking masks having the blocking areas and the passing areas thereof featuring the mask pattern of the mask set with corresponding displacements along one or more shifting directions.

9. The imaging method according to claim 8, wherein the mask patterns comprise a chessboard pattern with the blocking areas and the passing areas of rectangular shape being alternated along two perpendicular directions.

10. The imaging method according to claim 8, wherein the mask patterns comprise at least one strips pattern with the blocking areas and the passing areas having a strip shape extending along a common direction.

11. The imaging method according to claim 10, wherein the blinking masks are defined by corresponding matrices of cells each having a plurality of rows and a plurality of columns, the common direction of the blocking areas and of the passing areas extending along the rows, the columns, a first diagonal and/or a second diagonal of the matrices.

12. The imaging method according to any claim 8, wherein the imaging method comprises, for each of the mask sets:
providing, by the computing system an indication of the shifting directions and an indication of a shifting stride and of a shifting number for each of the shifting directions,
providing, by the computing system, a seed mask of the blinking masks of the mask set, and
generating, by the computing system the other blinking masks of the mask set by shifting the seed mask along each of the shifting directions by the shifting stride for the shifting number of the shifting direction.

13. The imaging method according to claim 8, wherein the imaging method comprises:
receiving, by the computing system an indication of an accuracy level for imaging the body-part, and
selecting, by the computing system, the mask sets, an extent of the blocking areas and of the passing areas of the blinking masks of each of the mask sets, the shifting directions of each mask set and/or the shifting stride for each of the shifting directions of each mask set according to the accuracy level.

14. The imaging method according to claim 1, wherein the imaging method comprises, for each of the input images:
determining, by the computing system a reduction region of the input image wherein the corresponding contrast particles are represented, and
setting, by the computing system an application of the blinking masks to the input image according to the reduction region thereof.

15. The imaging method according to claim 1, wherein the imaging method comprises:
filtering, by the computing system, any multiple determinations of different positions of each of the distinguishable contrast particles in the masked images of each of the input images.

16. (canceled)

17. A computer program product comprising a computer readable storage medium embodying a computer program, the computer program being loadable into a working memory of a computing system thereby configuring the computing system to perform the imaging method according to claim 1.

18. (canceled)

19. A computing system for imaging a body-part of a patient with an imaging method of ultrasound type, wherein the computing system comprises:
an input module for receiving a sequence of input images representative of any contrast particles of a contrast agent that are comprised in the body-part,
a generation module for generating a plurality of masked images from each of at least part of the input images by applying corresponding blinking masks thereto, each of the blinking masks alternating a plurality of blocking areas and a plurality of passing areas wherein the input image is masked and not masked, respectively.
a determination module for determining corresponding positions of any distinguishable ones of the contrast particles being distinguishable from any other ones of the contrast particles in each of the masked images, and
an output module for outputting analysis information of the body-part on the basis of the positions of the distinguishable contrast particles.

20. An imaging system of ultrasound type comprising the computing system according to claim 19 and an imaging probe for acquiring the sequence of input images, the computing system being coupled with the imaging probe for receiving the sequence of input images therefrom.

21. A medical method for imaging a body-part of a patient, wherein the medical method comprises:
administering a contrast agent to the patient,
acquiring a sequence of input images representative of any contrast particles of the contrast agent that are comprised in the body-part,
imaging the body-part according to the imaging method according to claim 1 thereby outputting the analysis information, and
performing a medical procedure relating to the body-part according to the analysis information.

22. The medical method according to claim 21, wherein the medical method is a diagnostic method comprising evaluating a health condition of the body-part according to the analysis information, a therapeutic method comprising treating the body-part according to the analysis information or a surgical method comprising operating the body-part according to the analysis information.
